(19) United States
(12) Patent Application Publication
(10) Pub. No.: US 2022/0227859 A1
(43) Pub. Date: Jul. 21, 2022

(54) METHOD TO TREAT TYPE 2 INFLAMMATION OR MAST-CELL DEPENDENT DISEASE

(71) Applicants: INSERM (INSTITUT NATIONAL DE LA SANTÉ ET DE LA RECHERCHE MÉDICALE), Paris (FR); UNIVERSITÉ PAUL SABATIER TOULOUSE III, Toulouse (FR); UNIVERSITY OF STANFORD, Stanford, CA (US)

(72) Inventors: Nicolas GAUDENZIO, Toulouse (FR); Stephen GALLI, Stanford, CA (US)

(21) Appl. No.: 17/611,231

(22) PCT Filed: May 15, 2020
(86) PCT No.: PCT/EP2020/063575

§ 371 Date: Nov. 15, 2021

(30) Foreign Application Priority Data
May 16, 2019 (EP) 19305625.6

(51) Int. Cl. C07K 16/28; A61P 37/08

(52) U.S. Cl. CPC C07K 16/28; A61P 37/08

(57) ABSTRACT

The present invention relates to the treatment of type 2 inflammation or mast-cell dependent disease. The inventors showed that, when exposed to domestic allergic alarms, activation of TRPV1+Tac1+ nociceptor-MRGPRB2+ MC sensory clusters might represent a key early event controlling the development of frequent mast cell-dependent allergic disorders. The human ortholog of MRGPRB2 (MRGPRX2) can thus be a good target to treat type 2 inflammation or mast cell-dependent disorders. Thus, the present relates to a MRGPRX2 inhibitor for use in the treatment of a type 2 inflammation or mast cell-dependent disorders in a subject in need thereof.

### FIELD OF THE INVENTION

The present invention relates to a MRGPRX2 inhibitor for use in the treatment of a type 2 inflammation or mast cell dependent disease in a subject in need thereof.

### BACKGROUND OF THE INVENTION

Altered tissue sensitivity to environmental triggers is thought to contribute to the development of allergic diseases, frequently starting with childhood type 2 (allergic) skin inflammation (1). Recent evidences have shown that nociceptive sensory neurons (i.e., nociceptors) can modulate the activation of innate immune cells (2-9), but it remains unclear whether specialized neuro-immune cross-talks play a role in the development of allergic inflammation. Here we show that house dust mites (HDM), common indoor allergens suspected to be involved in many allergic disorders (10), are potent activators of a defined subpopulation of peptidergic nociceptors expressing the transient receptor potential cation channel subfamily V member 1 (TRPV1) and secreting the cationic neuropeptide substance P (SP). Activated skin nociceptors induce the degranulation of contiguous mast cells (MCs) via the newly-described receptor for cationic molecules MRGPRB2 (11) and trigger the development of the pathological features associated with a model of allergic skin inflammation. In vivo ablation of TRPV1+ neurons, deletion of the gene Tac1 (12) (which encodes the SP precursor) or genetic inactivation of MRGPRB2 significantly reduce the development of skin disease and preserve optimal skin barrier architecture. Finally, real-time multiphoton microscopy of living mice reveals that the majority of TRPV1+ neuron projections in the skin form functional knots with MCs and that the presence of HDM in the skin dermis is sufficient to trigger the sequential activation of both cell types.

### SUMMARY OF THE INVENTION

The data of the inventors indicate that, when exposed to domestic allergic alarms, activation of TRPV1+Tac1+ nociceptor-MRGPRB2+ MC sensory clusters might represent a key early event controlling the development of frequent allergic disorders. Moreover the inventors demonstrate under homeostatic conditions, skin and peritoneal mast cells (innate immune cells thought to be involved in allergic diseases, including AD) in mice specifically express MRGPRB2, a receptor for cationic molecules from the Mas-related G protein-coupled receptors family. MRGPRX2 or MRGPRB2-mediated activation of mast cells can result in a remarkably fast degranulation dynamics, which is associated with the development of rapid and localized mast cell-dependent inflammation. Thus, the human ortholog of MRGPRB2 (MRGPRX2) can be a good target to treat type 2 inflammation or mast cell dependent disease.

Thus, the present invention relates to a MRGPRX2 inhibitor for use in the treatment of a type 2 inflammation or mast cell dependent disease in a subject in need thereof. Particularly, the invention is defined by its claims.

### DETAILED DESCRIPTION OF THE INVENTION

The present invention also relates to a MRGPRX2 binding molecule for use in the treatment of type 2 inflammation in a subject in need thereof.

The present invention relates to a MRGPRX2 inhibitor for use in the treatment of a type 2 inflammation in a subject in need thereof. In a particular embodiment the invention relates to a MRGPRX2 inhibitor for use in the treatment of a type 2 inflammation in a subject in need thereof.

As used in the invention, the term “type 2 inflammation” encompasses type 2 inflammation of the skin, type 2 allergy and type 2 allergy of the skin.

In a particular embodiment, the invention relates to a MRGPRX2 inhibitor for use in the treatment of a type 2 inflammation of the skin in a subject in need thereof.

In a particular embodiment, the invention relates to a MRGPRX2 inhibitor for use in the treatment of type 2 allergy in a subject in need thereof.

In a particular embodiment, the invention relates to a MRGPRX2 inhibitor for use in the treatment of type 2 allergy of the skin in a subject in need thereof.

The present invention also relates to a MRGPRX2 binding molecule for use in the treatment of mast cell dependent disease in a subject in need thereof.

The present invention also relates to a MRGPRX2 inhibitor for use in the treatment of mast cell dependent disease in a subject in need thereof.

In a particular embodiment the invention relates to a MRGPRX2 binding molecule for use in the treatment of mastocytosis in a subject in need thereof.

In a particular embodiment the invention relates to a MRGPRX2 inhibitor for use in the treatment of mastocytosis in a subject in need thereof.

As used herein, the term “mast cell” refers to a hematopoietic-derived cell that mediates hypersensitivity reactions. Mast cells are characterized by the presence of cytoplasmic granules (histamine, chondroitin sulfate, proteases) that mediate hypersensitivity reactions, high levels of the receptor for IgE (FceRI), and require, among other factors, stem cell factor and IL3 (cytokines) for development. In healthy individuals, mature mast cells are not found in the circulation, but reside in a variety of tissues throughout the body.

As used herein, the term “mast cell dependent disease” refers to any inflammation characterized by pathological mast cell proliferation and/or activation (e.g. degranulation or cytokines production). Examples of mast cells inflammation include any disease selected from the group consisting of mast cell activation syndrome (MCAS); mastocytosis; idiopathic urticaria; chronic urticaria; atopic dermatitis; idiopathic anaphylaxis; Ig-E and non Ig-E mediated anaphylaxis; angioedema; allergic disorders; irritable bowel syndrome; mastocytic gastroenteritis; mastocytic colitis; fibromyalgia; kidney fibrosis; atherosclerosis; myocardial ischemia; hypertension; congestive heart failure; pruritus; chronic pruritus; pruritus secondary to chronic kidney failure; heart, vascular, intestinal, brain, kidney, liver, pancreas, muscle, bone and skin conditions associated with mast cells; CNS diseases such as Parkinson's disease and Alzheimer's disease; metabolic diseases such as diabetes; sickle cell disease; autism; chronic fatigue syndrome; lupus; chronic lyme disease; interstitial cystitis; multiple sclerosis; cancer; migraine headaches; psoriasis; eosinophilic esophagitis; eosinophilic gastroenteritis; Churg-Strauss syndrome; hypereosinophilic syndrome; eosinophilic fasciitis; eosinophilic gastrointestinal disorders; chronic idiopathic urticaria; myocarditis; Hirschsprung's-associated enterocolitis; postoperative ileus; wound healing; stroke; transient ischemic attack; pain; neuralgia; peripheral neuropathy; acute coronary syndromes; pancreatitis; dermatomyositis; fibrotic skin diseases; pain associated with cancer; ulcerative colitis; inflammatory bowel disease; radiation colitis; celiac disease; gluten enteropathy; radiation cystitis; painful bladder syndrome; hepatitis; hepatic fibrosis; cirrhosis; rheumatoid arthritis; lupus erythematosus; and vasculitis.

As used herein, the term “mastocytosis” has its general and describes a group of disorders in which pathologic mast cells accumulate in tissues. In particular, the term includes cutaneous mastocytosis, systemic mastocytosis (indolent or advanced) and mast cell leukemia.

As used herein, the term “mast cell activation syndrome” or “MCAS” has its general meaning in the art and encompasses a collection of clinical signs and symptoms resulting from the inappropriate activation of mast cells, wherein no proliferation or otherwise accumulation of mast cells is observed.

As used herein the terms “MRGPRX2 binding molecule” denotes molecules or compound which can bind the MRGPRX2 protein. The binding protein comprises a “binding domain” As used herein, the term “binding domain”, i.e. an amino acid sequence region that preferentially binds to the target molecule under physiological conditions. Binding molecule include binding protein such as antibody but as well as any other proteins potentially capable of binding a given target molecule that typically include but are not limited to protein ligands and receptors, aptamer, polypeptide.

In one embodiment, the MRGPRX2 binding molecule according to the invention is a low molecular weight compound, e.g. a small organic molecule (natural or not), antibody, aptamer, or polypeptide.

In some embodiment, the MRGPRX2 binding molecule is a mast cell depleting molecule.

In other words, in some embodiment, the MRGPRX2 binding molecule is a depleting molecule who lead to deplete mast cells.

In some embodiment, the MRGPRX2 binding molecule is MRGPRX2 inhibitor.

The term “binding” as used herein refers to a direct association between two molecules, due to, for example, covalent, electrostatic, hydrophobic, and ionic and/or hydrogen-bond interactions, including interactions such as salt bridges and water bridges. In particular, as used herein, the term “binding” in the context of the binding of a binding protein (e.g. an antibody) to a predetermined target molecule (MRGPRX2) typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less.

As used herein, the term “MRGPRX2” for “Mas-related G-protein coupled receptor member X2” has its general meaning in the art and refers to a protein that in humans is encoded by the MRGPRX2 gene. Agonists are a broad panel of cationic molecules, such as endogenous neuropeptides like substance P or vasoactive intestinal peptide, antimicrobial peptides like LL-37, but also broad panel of clinically-approved drugs such as gyrase inhibitors like ciprofloxacine and non-steroidal neuromuscular blocking agents like atracurium as well as vancomycin. Activation of MRGPRX2 leads to mast cell degranulation with subsequent pseudo-allergic reactions. The Ensembl number of the human gene is ENSG00000183695. The mouse ortholog is the Mrgprb2, used as prove of concept in the present application.

The invention also relates to an MRGPRB2 (the analogue of MRGPRX2) inhibitor for use in the treatment of a type 2 inflammation or mast cell-dependent disease in a mouse.

The terms “MRGPRX2 inhibitor” denotes molecules or compound which can inhibit the activity of the protein (e.g. inhibit the MRGPRX2-dependent mast cell degranulation) or a molecule or compound which destabilizes the protein.

The term “MRGPRX2 inhibitor” also denotes inhibitors of the expression of the gene coding for the protein.

In order to test the functionality of a putative MRGPRX2 inhibitor a test is necessary. For that purpose, to identify MRGPRX2 inhibitors, one can use cell lines transfected with a plasmid coding for MRGPRX2 or its mouse ortholog Mrgprb2 to enable the selective expression of the proteins MRGPRX2 or MRGPRB2 on the cell surface. Those transfected cells can then be used as a reporter system to screen potential MRGPRX2/MRGPRB2 inhibitory molecules as described in the patent WO2016019246A1. One can also use human or mouse cells expressing naturally MRGPRX2 or MRGPRB2 (e.g., primary mast cells or in vitro-derived mast cells from hematopoietic progenitors).

As used herein, the term “subject” denotes a mammal, such as a rodent like a mouse, a feline, a canine, and a primate. Particularly, the subject according to the invention is a human.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein the term “type 2 inflammation” means an inflammatory reaction which is characterized by the overproduction of Th2- or type 2 innate lymphoid cells (ILC2)-derived cytokines like IL4 or IL13, including those that result from an overproduction or bias in the differentiation of T-cells into the Th2 subtype or an abnormal number of ILC2.

As used the terms type 2 inflammation of the skin denotes a sub-group of type 2 immune response-associated with the development of skin pathology.

Type 2 inflammation includes, for example, anaphylactic hypersensitivity, asthma, allergic rhinitis, atopic dermatitis, vernal conjunctivitis, eczema, IgE-mediated urticarial, food allergies, IgE-mediated anaphylaxis, esophagus eosinophilic or oesophagitis.

Type 2 allergy of the skin includes, for example, atopic dermatitis or eczema.

As used herein, terms such as “Th2 cell” and/or “Th2 phenotype” and all grammatical variations thereof refer to a differentiated CD4⁺ T helper cell that expresses IL-4.

In a particular embodiment, the MRGPRX2 inhibitor according to the invention is used for the treatment of atopic dermatitis.

In a particular embodiment, the MRGPRX2 inhibitor is used in combination with any immune adjuvant inducting and/or promoting Th1 cell differentiation (e.g. Th1 adjuvant).

In a particular embodiment, the MRGPRX2 binding molecule is used in combination with any immune adjuvant inducting and/or promoting Th1 cell differentiation (e.g. Th1 adjuvant).

Thus, the invention also relates to a i) MRGPRX2 inhibitor, and ii) a Th1 adjuvant, as a combined preparation for simultaneous, separate or sequential use in the treatment of a type 2 inflammation or mast cell-dependent disease in a subject in need thereof.

Thus the invention also relates to a i) MRGPRX2 binding molecule, and ii) a Th1 adjuvant, as a combined preparation for simultaneous, separate, sequential use in the treatment of a type 2 inflammation or mast-cell dependent disease in a subject in need thereof.

As used herein, the Th1 adjuvants are selected in the group consisting in but not limited to IL-12, LPS, Complete Freund's adjuvant, Aluminium salts like Alum, CpG, squalene (see for example Coffman R L et al., Immunity 2010).

In one embodiment, the inhibitors according to the invention may be a low molecular weight compound, e.g. a small organic molecule (natural or not).

The term “small organic molecule” refers to a molecule (natural or not) of a size comparable to those organic molecules generally used in pharmaceuticals. The term excludes biological macromolecules (e.g., proteins, nucleic acids, etc.). Preferred small organic molecules range in size up to about 10000 Da, more preferably up to 5000 Da, more preferably up to 2000 Da and most preferably up to about 1000 Da.

In one embodiment, the MRGPRX2 binding molecule according to the invention is an antibody

In one embodiment, the inhibitor according to the invention (inhibitor of MRGPRX2) is an antibody.

Antibodies or directed against MRGPRX2 can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against MRGPRX2 can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g., U.S. Pat. No. 4,946,778) can be adapted to produce anti-MRGPRX2 single chain antibodies. Compounds useful in practicing the present invention also include anti-MRGPRX2 antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to MRGPRX2.

Humanized anti-MRGPRX2 antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

In some embodiment, for this invention, neutralizing antibodies of MRGPRX2 is selected.

In a particular embodiment, the anti-MRGPRX2 antibody according to the invention may be the 5H6A12 or K.137.7 antibodies as send by Thermofisher.

In some embodiment, for this invention, mast cell depleting antibodies of MRGPRX2 is selected.

As used herein, the term “mast cell depleting antibody” refers to antibody which lead to depletion of the population of mast cell.

In other words, in some embodiment, the antibody of MRGPRX2 is a depleting antibody who lead to deplete mast cells.

Thus, in some embodiment, the antibody of MRGPRX2 is a mast cell depleting antibody or neutralizing antibody.

In another embodiment, the antibody according to the invention is a single domain antibody against MRGPRX2. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In one embodiment, the compound according to the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamer of MRGPRX2 is selected.

In one embodiment, the compound according to the invention is a polypeptide.

In a particular embodiment the polypeptide is an antagonist of MRGPRX2 and is capable to prevent the function of MRGPRX2. Particularly, the polypeptide can be a mutated MRGPRX2 protein or a similar protein without the function of MRGPRX2. In this case, the mutated version of the MRGPRX2 protein is used as a decoy receptor.

In one embodiment, the polypeptide of the invention may be linked to a “cell-penetrating peptide” to allow the penetration of the polypeptide in the cell.

The term “cell-penetrating peptides” are well known in the art and refers to cell permeable sequence or membranous penetrating sequence such as penetratin, TAT mitochondrial penetrating sequence and compounds (Bechara and Sagan, 2013; Jones and Sayers, 2012; Khafagy el and Morishita, 2012; Malhi and Murthy, 2012).

The polypeptides of the invention may be produced by any suitable means, as will be apparent to those of skill in the art. In order to produce sufficient amounts of polypeptide or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known.

When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E. coli.

In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 Daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomerular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In another embodiment, the MRGPRX2 inhibitor according to the invention is an inhibitor of MRGPRX2 gene expression.

Small inhibitory RNAs (siRNAs) can also function as inhibitors of MRGPRX2 expression for use in the present invention. MRGPRX2 gene expression can be reduced by contacting a subject or cell with a small double stranded RNA (dsRNA), or a vector or construct causing the production of a small double stranded RNA, such that MRGPRX2 gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of MRGPRX2 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of MRGPRX2 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of MRGPRX2 gene expression can be prepared by known methods. These include techniques for chemical synthesis such as, e.g., by solid phase phosphoramidite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing MRGPRX2. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, the antisense oligonucleotide, siRNA, shRNA or ribozyme nucleic acid sequence is under the control of a heterologous regulatory region, e.g., a heterologous promoter. The promoter may be specific for Muller glial cells, microglia cells, endothelial cells, pericyte cells and astrocytes For example, a specific expression in Muller glial cells may be obtained through the promoter of the glutamine synthetase gene is suitable. The promoter can also be, e.g., a viral promoter, such as CMV promoter or any synthetic promoters.

In another embodiment, the invention relates to a method for treating a type 2 inflammation or mast cell-dependent disease comprising administering to a subject in need thereof a therapeutically effective amount of a MRGPRX2 inhibitor.

By a “therapeutically effective amount” is meant a sufficient amount of the inhibitor of MRGPRX2 to treat a type 2 inflammation at a reasonable benefit/risk ratio applicable to any medical treatment.

### Therapeutic Composition

Another object of the invention relates a therapeutic composition comprising a MRGPRX2 binding molecule according to the invention for use in the treatment of a type 2 inflammation or mast cell-dependent disease in a subject in need thereof.

Another object of the invention relates a therapeutic composition comprising a MRGPRX2 inhibitor according to the invention for use in the treatment of a type 2 inflammation or mast cell-dependent disease in a subject in need thereof.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

Pharmaceutical compositions of the present invention may comprise a further therapeutic active agent. The present invention also relates to a kit comprising an inhibitor according to the invention and a further therapeutic active agent, particularly an anti-inflammatory compound.

For example, these agents can be nonsteroidal anti-inflammatory drugs like aspirin, ibuprofen, and naproxen, β-agonists, corticoids, anti-histaminics, antileukotrienne, antibodies anti-IgE, anti-IL5 or anti-IL4Ra/IL13Ra (like the dupilumab) (see for example Akdis C A, 2012).

The invention will be further illustrated by the following figures and examples. However, these examples and figures should not be interpreted in any way as limiting the scope of the present invention.

### FIGURES

FIG. 1. Genetic inactivation of MRGPRB2 protects from pathological features development in a model of allergic skin inflammation. Mrgprb2^{mut} and Mrgprb2^{+/+} mice were treated with D. farinae+SEB to induce allergic skin inflammation. A, Clinical scores (0-12). B, Epidermal thickness (μm). C,D, Number of eosinophils (C) and neutrophils (D) in skin sections. E, fluorescence analysis of filaggrin staining. Bars=100 μm. Each open circle=one mouse; Mean±SEM (A); Black bars: Mrgprb2^{+/+} mice, grey bars: Mrgprb2^{mut}. Mean+SEM; two-tailed, unpaired t-test, *P<0.05 **P<0.01 ***P<0.001. Data pooled from 3 independent experiments performed with at least 6 mice per group.

### EXAMPLE

Material & Methods

Mice.

Four- to 8-weeks-old C57BL/6J and Tac1^{-/-} mice were purchased from Charles River or the Jackson Laboratory, both male and female mice were used in experiments. MrgprB2^{mut} mice (in which MrgprB2 is genetically inactivated by mutation¹¹) and Pirt-GCaMP3 mice (in which a genetically encoded calcium tracer [GCaMP3] is driven by the Pirt promoter into sensory neuron²⁸) were provided by X. Dong and both male and female mice were used in experiments. The two MC-deficient Kit^{W-Sh/W-sh} and Cpa3-cre⁺; Mcl-1^{fl/fl} mice have been described previously^{20,27}, both male and female mice were used in experiments. Mice were bred and housed in the local animal facilities of CREFRE (Toulouse, France) and Stanford University (CA, USA), and littermate control mice were used in all experiments.

Animal Study Approval.

All animal care and experimentation were conducted in France and in the USA. Experimentations conducted in USA (Galli Lab, Stanford University, Calif.) were in compliance with the guidelines of the National Institutes of Health (NIH) and the Institutional Animal Care and Use Committee of Stanford University. Experimentations conducted in France (Gaudenzio Lab, INSERM, University of Toulouse) were in compliance with the guidelines of the European Union (86/609/EEC) and the French Committee of Ethics (87/848) policies and with the specific approval from the local ministry-approved committee on ethics in animal experimentation (Ethics Committee UMS006 CEEA-122, project No. 13283 2018031416055447V3).

Reagents and Antibodies.

Sodium citrate, Bovine Serum Albumin (BSA), DMSO, saponin, Capsaicin, Resiniferatoxin and Staphylococcal Enterotoxin B (SEB) were from Sigma-Aldrich. HDM extracts of the strain Dermatophagoides farinae were purchased from Greer Laboratories. The following antibodies were obtained from Covance: anti-Keratin (K) 14, anti-K6, anti-K10, anti-loricrin and anti-filaggrin. Anti-Claudin 1 was from Abcam. Alexa594-conjugated goat anti-rabbit, Alexa488-conjugated avidin and DAPI were from Life Technologies Invitrogen. The following reagent and antibodies were from eBioscience Thermofisher Scientific: CellTrace™ CFSE Cell Proliferation Kit, anti-CD4-APC, anti-IL4-PE, anti-IL-5-PE, anti-IL-13-PE, anti-IFNγ-PE.

Model of Allergic Skin Inflammation.

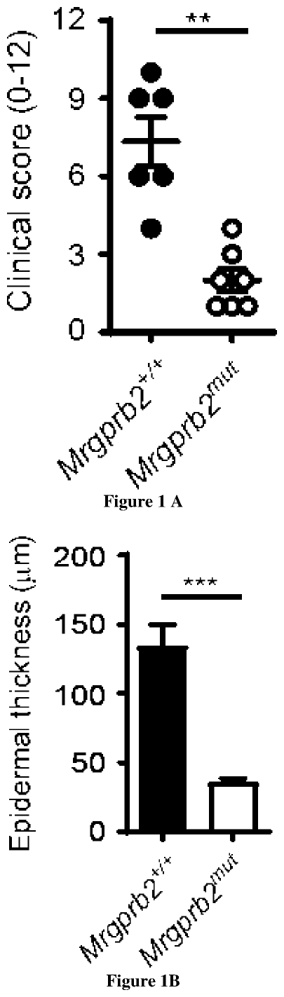

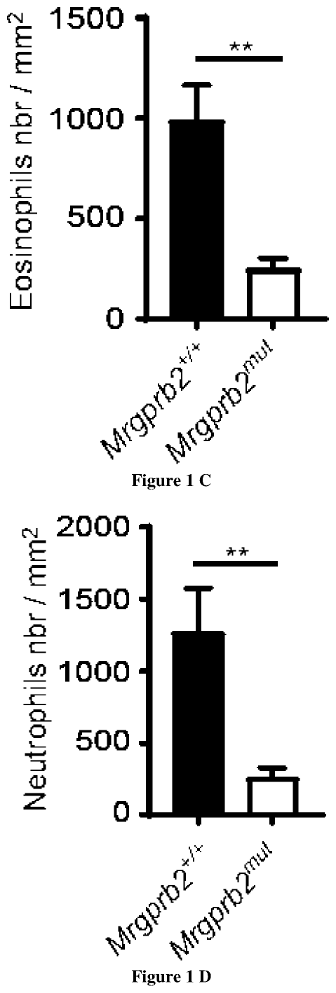

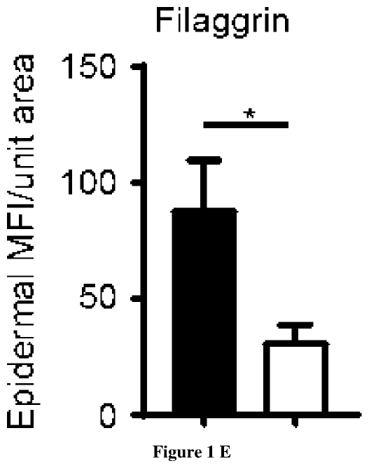

Allergic skin inflammation was induced as previously described^{13,14} (described in Extended data FIG. 1). Briefly, back skin was shaved and a solution of 500 ng of Staphylococcal enterotoxin B (SEB, Sigma-Aldrich) and of 10 μs of Dermatophagoides farinae extract (HDM, Greer Laboratories) in PBS was applied on a gauze pad placed on the shaved back and occluded with a Tegaderm™ Transparent Dressing (3M HealthCare). Three days later, the gauze pads were replaced. Mice were monitored on a daily basis and if a mouse removed the bandage, a new dressing was immediately applied on this mouse and all the other mice within the same experiment (so that they receive the same treatment and equal amount of antigens). Four days later, dressings were removed and mice were kept without treatment for the next week. This “3+4” days pattern of treatment was repeated two more times, so that the mice were subjected to three cycles of such treatment. Two days after the last cycle of treatment, the mice were euthanized and back skin specimens corresponding to the treated areas were obtained for analyzes. This model has been efficiently used in both INSERM Toulouse and Stanford University and key experiments have been repeated in both animal facilities.

Intracellular Flow Cytometry of D. farinae-Specific CD4⁺ T Cells.

Following induction of allergic skin inflammation, spleens from vehicle-treated or D. farinae+SEB-treated mice were harvested and dissociated to obtain a suspension of cells. 200,000 splenic cells were stained with CFSE 7 minutes at 37° C. and incubated for 5 days with 10 μg/ml of D. farinae in RPMI 1640 supplemented with 10% FCS, GlutaMAX-I, sodium pyruvate, 2-mercaptoethanol, ciprofloxacin. Intracellular cytokines were analyzed by gating on proliferating (CFSE^{low}) CD4⁺ T cells after 5-hour restimulation with phorbol 12-myristate 13-acetate (50 ng/mL, Sigma) and ionomycin (1 mg/mL, Sigma) in the presence of GolgiStop (BD Pharmingen). Cells were fixed, permeabilized (0.1% saponin in PBS 0.5% BSA), and stained with antibodies directed against mouse IL-4, IL-5, IL-13 and IFN-γ. Flow cytometric data were acquired on a BD FACSCanto cytometer and were analyzed using FlowJo software (Tree Star, Inc, Ashland, Ore).

Skin Section Preparation, Histology, Immunofluorescence and Confocal Microscopy.

Mouse back skin (1-2 cm²) samples were fixed in 10% formalin and embedded in paraffin. Four-micrometer-thick sections were stained with H&E, and photographs were taken using a Nikon H600L microscope and analyzed with NIS-Elements imaging Software. All sections were “coded” so the evaluator was not aware of their identity, as previously described¹³. For immunostaining of mouse specimens, 4-μm-thick sections were pretreated using a heat-induced epitope retrieval method¹³ in 10 mM sodium citrate buffer (pH 6.0), then permeabilized for 30 minutes in PBS supplemented with 0.5% BSA and 0.1% saponin. Permeabilized skin sections were incubated overnight at 4° C. with primary antibodies, extensively washed, and incubated with appropriate secondary antibodies for 2 hours at room temperature in the dark. Images 1024×1024 pixels were acquired using a Zeiss LSM780 and LSM710 Meta inverted confocal laser-scanning microscopes. Images were processed using Zen software (Zeiss). Epidermal K6, K17, Claudin 1, Filaggrin, loricrin and e-cadherin mean fluorescence intensities were analyzed using the “measurement function” of ImageJ software on randomly chosen epidermal areas of identical size (i.e., same total number of pixels).

Dorsal Root Ganglia (DRG) Dissociation, Culture, and Ca²⁺ Imaging.

DRG neurons from all spinal levels were collected in ice-cold Dulbecco's modified Eagle's medium (DMEM)/F-12 supplemented with 10% FBS, penicillin (100 U/mL), and streptomycin (100 μg/mL). DRGs were digested with a mixture of dispase (5 mg/ml) and collagenase type I (1 mg/ml) enzyme at 37° C. for 45 minutes. After dissociation, cells were spun at 300 g and re-suspended in media before being plated on glass coverslips coated with poly-D-lysine (0.5 mg/ml) and laminin (10 μg/ml, Invitrogen). DRGs were cultured in media supplemented with 50 ng/mL NGF at 37° C. overnight (12-24 hrs) before experimentation. Cells were imaged in calcium imaging buffer (CM; 10 mM HEPES, 1.2 mM NaHCO₃, 130 mM NaCl, 3 mM KCl, 2.5 mM CaCl₂, 0.6 mM MgCl₂, 20 mM glucose, and 20 mM sucrose at pH 7.4 and 290-300 mOsm). To monitor changes in intracellular [Ca²⁺] ([Ca²⁺]ᵢ), cells were loaded with Fura2-AM for 30 minutes in the dark at 37° C. in CIB just prior to imaging. Emission at 520 nm was monitored after excitation at 340 nm (Ca²⁺ bound) or 380 (unbound). Cells were imaged for 20 seconds to establish a baseline before compounds were added. At the end of every imaging trial, 50 mM KCl was added as a positive control. Cells were identified as responding if the intracellular [Ca²⁺] rose by either 50% compared to baseline or 50% compared to the [Ca²⁺]ᵢ change assayed during addition of 50 mM KCl (neurons only). Damaged, detached, high-baseline, and motion-activated cells were excluded from analysis. Those experiments were performed in the Dong Lab at John Hopkins University (and confirmed in the Gaudenzio Lab at INSERM, Toulouse France, data not shown) in compliance with John Hopkins University ethical guidelines.

In Vivo Two-Photon Microscopy of Living Mice.

Experiments were conducted as previously described^{20,27}. In brief, 8 μg of Av.SRho in 20 μl of PBS were injected i.d. into the ear pinna of Pirt-GCaMP3 mice. 1 week later, mice were injected i.d. with vehicle, 1 μM capsaicin, 1 μg D. farinae and 50 ng SEB (used alone or in combination) in a final volume of 20 μl then placed under the two-photon microscope on a custom-built 3-D printed mouse platform, anesthesia was maintained by a mixture of Isoflurane/O2 and the animal's ear pinna was kept at 36° C. using a heating pad system. The fluorescence corresponding to Av.SRho⁺ MC granule structures or GCaMP3⁺ skin neurons were measured using a Prairie Ultima IV two-photon microscope (Spectra Physics Mai Tai HP Ti:sapphire laser, tunable from 690 to 1040 nm). Images were acquired in 3-D up to 100-150 μm depth, with 20× Olympus XLUM Plan Fl N.A. 0.95 water-immersion objective and a software zoom setting of 1 or 3 (8 bits/pixel 1024×1024, scaling x=0.228 μm, y=0.228 μm, z=0.5 μm). Modeling and analysis of fluorescent signals were performed using untreated image sequences, as previously described, using Imaris software (Bitplane) and Image J software version Fiji, respectively. For the time-lapse experiments described in Extended data video 1, the fluorescence corresponding to Av.SRho⁺ MC granule structures or GCaMP3⁺ skin neurons were monitored over time after injection of vehicle (PBS) control or 1 μM TRPV1 agonist capsaicin.

Automated Computational Analysis of the Minimum Distance Between Mast Cells and Neurons in the Dermis of Living Mice.

An example of analysis is shown in Extended data FIG. 8. 1) 3D high resolution images were taken using a Prairie Ultima IV two-photon microscope as described above. The following steps have been automated in the software Imaris Bitplane version 9.2. 2) Hair follicles autofluorescent signals were modeled into matched 3D objects using the isosurface algorithm. 3) Autofluorescent signals corresponding to the generated isosurfaces were depleted from GCaMP3 fluorescence detection channel such as the hair follicles were no longer detectable in that particular channel. 4) The filament tracer algorithm was applied in the GCaMP3 fluorescence detection channel in order to precisely trace the trajectories and exact shapes of GCaMP3 fluorescent signals. Filament traces are then converted into fluorescent signals into a new fluorescent channel. 5) Those newly generated fluorescent signals were modeled into matched 3D objects using the isosurface algorithm. The distance transformation algorithm was applied to those new isosurfaces resulting in the generation of a new distance transformation channel. 6) Av.SRho fluorescent signals were modeled into matched 3D objects using the isosurface algorithm. The intensity minimum (i.e., distance minimum in μm) to the distance transformation channel (i.e., modeled sensory neurons) was calculated for each Av.SRho⁺ isosurfaces of at least 5 μm of diameter (corresponding to Av.SRho⁺ MC cellular bodies and excluding small exteriorized Av.SRho⁺ granules structures). 7) Results per field of view were generated into separated Excel sheets. The exact same procedure was automatically applied to all analyzed 3D images.

Statistics.

Statistical tests were performed with the software Prism 6 (GraphPad Software). Two-tailed unpaired Student's t tests were performed on samples with different variances as noted in the respective figure legends. A P value of less than 0.05 was considered statistically significant.

Results

We employed a mouse model of allergic skin inflammation that uses repeated epicutaneous exposures to two antigens frequently found in the lesional skin of most atopic dermatitis (AD) patients, the HDM strain Dermatophagoides farinae (D. farinae) and the bacteria exotoxin Staphylococcal enterotoxin B ([SEB] from Staphylococcus aureus) (data not shown), to induce a severe dermatitis associated with histopathological features, systemic D. farinae-specific Th2 response (data not shown) and a global gene expression pattern similar to that in human AD^{13,14}. Clinical findings have shown that, compared to healthy people, the serum and skin of AD patients exhibit elevated levels of neuropeptides (e.g., SP) and that the amounts detected correlate with the severity of the disease^{15,16}. Recent work has shown that a unique subpopulation of TRPV1⁺, TRPA1⁻ peptidergic nociceptors is highly positive for Tac1¹², the gene encoding the SP precursor¹⁷. Using publicly available gene expression data¹⁸, we mapped the expression of Trpv1, Tac1 and Trpa1 among different mouse tissues. We confirmed that Trpv1, Tac1 and Trpa1 genes were highly (if not exclusively in the case of Trpv1 and Trpa1) expressed in dorsal root ganglia (DRG), with a weak expression of Tac1 in the central and the enteric nervous systems (data not shown). In line with those observations, we analyzed the expression of SP in whole-mounted back skin biopsies from C57BL/6J wild type (WT) and found that SP expression was restricted to PGP9.5⁺ cutaneous neuronal fibers (data not shown). To analyze the role of SP in the development of allergic skin inflammation, we treated WT and Tac1^{-/-} mice with D. farinae and SEB and assessed the development of key AD-associated pathological features¹³.

Compared to vehicle control-treated WT mice, D. farinae and SEB-treated WT mice developed macroscopic skin lesions, increase in epidermal thickness, strong infiltration of eosinophils and neutrophils (data not shown), and a profound alteration of filaggrin protein expression (data not shown), a key component of the stratum corneum involved in skin barrier function and suspected to be linked to human AD. Moreover, they also showed increase in expression of keratin (K)-6 (a marker of inflammatory stress in keratinocytes, data not shown) and additional abnormalities in other tested epidermal proteins, such as the claudin-1, K-14 and K-10, but not loricrin or e-cadherin (data not shown). Conversely, D. farinae and SEB-treated Tac1^{-/-} mice were mostly protected from disease development with substantial reduction of skin lesions development, histological abnormalities, infiltration of immune cells and with unaltered skin barrier architecture (data not shown). Taken together, our data strongly suggest that Tac1 gene and its product the SP are restricted to neuronal compartments and that the expression of Tac1 is required for the development of all pathological features associated with a model allergic skin inflammation.

To analyze the role of nociceptors in the development of allergic skin inflammation, we treated systemically WT mice with resiniferatoxin (RTX) to selectively ablate TRPV1⁺ nociceptors^{2,3,19} (data not shown). We subsequently induced allergic skin inflammation in RTX-treated mice vs. DMSO-treated control mice and assessed the development of pathological features¹³. Compared to DMSO-treated control mice, and in line with the results obtained in Tac1^{-/-} mice, RTX-treated mice showed a strong reduction of skin lesions development and tested pathological features (data not shown), with restored filaggrin organization and decreased expression of the stress marker K-6 (data not shown). These data strongly suggest that TRPV1⁺ nociceptors are required for the full development of a model allergic skin inflammation.

The primary function of nociceptors is to detect potentially damaging stimuli and initiate appropriate behavioral responses (e.g., removal or scratching). We thus investigated whether the two AD-associated antigens used in our model (i.e., D. farinae and SEB) could be directly detected by ex vivo cultured DRG neurons. We found that ng/ml concentrations of the domestic strain of HDM D. farinae (used alone or in combination with SEB) triggered a robust increase in intracellular calcium levels in a subpopulation of DRG neurons that also responded to the TRPV1 agonist, capsaicin (data not shown). However, the S. aureus-derived exotoxin SEB induced only a weak (barely detectable in some experiments) calcium influx in ex vivo DRG neurons (data not shown). In accordance with our previous observation in vivo (data not shown), we found D. farinae (alone or in combination with SEB) also trigger the secretion of SP from ex vivo cultured DRG neurons. These data indicate that common allergenic environmental alarms, that are suspected to play a pivotal role in various allergic disorders, can directly trigger the activation of a subset of TRPV1⁺ Tac1^{+/+} (SP-producing) peptidergic nociceptors.

Recent reports^{11,20-24} have shown that MRGPRB2 in mice (and its human ortholog MRGPRX2) are the MC-restricted receptors for several cationic substances, including the neuropeptide SP^{11,25}. Moreover, we previously reported that skin MCs activation by intradermal (i.d.) injection of recombinant SP exhibited specific dynamics and features of cytoplasmic granule secretion that were associated with the rapid induction of localized MRGPRB2- and MC-dependent inflammation²⁰. We therefore used both Kit-dependent Kit^{W-Sh/W-Sh} (data not shown) and Kit-independent Cpa3-cre⁺; Mc11^{fl/fl} (i.e., so called “hello kitty” mice, data not shown) MC-deficient mouse models²⁶ (data not shown) to study the role of MCs in the development of the pathological features associated with a model of allergic skin inflammation. Compared to their respective littermate controls, both MC-deficient mouse strains exhibited a marked reduction of skin lesions development and analyzed pathological features (data not shown). Importantly, we did not observe a reduction in the number of skin MCs either in Tac1^{-/-} mice or RTX-treated mice (data not shown), indicating that the observed reduced pathological features in those models could not be imputed to a lack/reduction in skin MC number. We next assessed whether functional MRGPRB2 could contribute to the development of allergic skin inflammation in the mouse model. Compared to Mrgprb2^{+/+} littermate controls mice, Mrgprb2^{mut} mice (i.e., in which the number of MCs is normal, but MRGPRB2 is genetically inactivated by mutation¹¹) were preserved from full disease development with substantial reduction of skin lesions, histological abnormalities, infiltration of immune cells and with intact skin barrier architecture (FIGS. 1 A, B, C, D and E). Taken together, these data strongly suggest that MCs expressing a functional MRGPRB2 contribute importantly to the development allergic skin inflammation in this model.

We recently reported a new method to specifically probe and monitor skin MC granules structures in living mice by longitudinal two-photon microscopy²⁷. We used this labeling method in the ear pinna of Pirt-GCaMP3 mice (in which a genetically encoded calcium tracer [GCaMP3] is driven by the Pirt promoter into sensory neurons²⁸) in order to track, simultaneously, the spatiotemporal dynamics of skin MC granule structures and TRPV1⁺ sensory neurons activation in living mice (data not shown). Following injection of vehicle control, we detected low basal levels of GCaMP3 fluorescence in neurons (in accordance with previous observations in unstimulated neurons ex vivo²⁸) and no degranulation of Sulforhodamine 101-labeled avidin⁺ (Av.SRho⁺) MCs (data not shown c). Upon i.d. injection of 1 μM of the TRPV1 agonist capsaicin, we detected a strong increase in GCaMP3 fluorescence levels revealing the presence of an abundant network of activated TRPV1⁺ nociceptors in the mouse dermis (data not shown). We also observed that, upon injection of the TRPV1 agonist, most of Av.SRho⁺ MCs exhibited a degranulated phenotype (i.e., Av.SRho⁺ granule structures exteriorized in the microenvironment, data not shown). When sequential images were recorded as a video for a period of 60 minutes, we noted that the increase in GCaMP3 fluorescence was preceding of ˜10-15 minutes the degranulation of cognate Av.SRho⁺ MCs, a timing potentially compatible with a phenomenon of TRPV1⁺ nociceptor-induced MC degranulation in vivo (data not shown). We next investigated nociceptor and MC activation after i.d. infusion of 1 μg D. farinae and 50 ng SEB either in combination or separately. We also detected a significant increase in GCaMP3 fluorescence in skin neurons and the presence of adjacent degranulated Av.SRho⁺ MCs (data not shown), a pattern of activation that strikingly resemble that obtained upon activation of TRPV1⁺ nociceptors by capsaicin. In line with our ex vivo observations in DRG neurons (data not shown), these data strongly suggest that the penetration into the dermis of D. farinae and SEB antigens leads to the sequential activation of sensory neurons and MCs in vivo. Finally, we set up an automated computer-assisted calculation method to perform an unbiased analysis of the spatial organization of TRPV1⁺ nociceptors and Av.SRho⁺ MCs in the dermis of living Pirt-GCaMP3 mice (data not shown). We found that the majority of Av.SRho⁺ MCs were either in close proximity (less than 25 μm of distance for 37% of them) or forming “synapse-like” contacts (for 25% of them) with functional TRPV1⁺ nociceptors in the skin (data not shown). Taken together, those data indicate that MCs and TRPV1⁺ nociceptors form functional cell-cell sensitive clusters that can be activated in the presence the antigenic alarms commonly found in skin lesions of AD patients, D. farinae and SEB. It is interesting to speculate that such narrow anatomical location in the mouse dermis might enable the accumulation of high enough levels of neuropeptide (e.g., SP) in order to reach the previously reported high activation threshold of MRGPRB2²⁹.

### CONCLUSION

We showed that two discrete neuronal and tissue-resident innate immune cell populations that share anatomical location in the skin, TRPV1⁺Tac1⁺ nociceptors and MRGPRB2⁺ MCs respectively, respond to common domestic allergens to profoundly disturb tissue homeostasis and drive the development of type 2 immunity-associated allergic skin inflammation. The present findings are adding important informations on how environmental triggers can be sensed by specialized cellular systems of different natures and could potentially spark the development of uncontrolled allergic disease³⁰.

### REFERENCES

Throughout this application, various references describe the state of the art to which this invention pertains. The disclosures of these references are hereby incorporated by reference into the present disclosure.
1 Zheng, T., Yu, J., Oh, M. H. & Zhu, Z. The atopic march: progression from atopic dermatitis to allergic rhinitis and asthma. Allergy Asthma Immunol Res 3, 67-73, doi:10.4168/aair.2011.3.2.67 (2011).
2 Riol-Blanco, L. et al. Nociceptive sensory neurons drive interleukin-23-mediated psoriasiform skin inflammation. Nature 510, 157-161, doi:10.1038/nature13199 (2014).
3 Kashem, S. W. et al. Nociceptive Sensory Fibers Drive Interleukin-23 Production from CD301b+ Dermal Dendritic Cells and Drive Protective Cutaneous Immunity. Immunity 43, 515-526, doi:10.1016/j.immuni.2015.08.016 (2015).
4 Cardoso, V. et al. Neuronal regulation of type 2 innate lymphoid cells via neuromedin U. Nature 549, 277-281, doi:10.1038/nature23469 (2017).
5 Baral, P. et al. Nociceptor sensory neurons suppress neutrophil and gammadelta T cell responses in bacterial lung infections and lethal pneumonia. Nat Med 24, 417-426, doi:10.1038/nm.4501 (2018).
6 Rankin, L. C. & Artis, D. Beyond Host Defense: Emerging Functions of the Immune System in Regulating Complex Tissue Physiology. Cell 173, 554-567, doi:10.1016/j.cell.2018.03.013 (2018).
7 Moriyama, S. et al. beta2-adrenergic receptor-mediated negative regulation of group 2 innate lymphoid cell responses. Science 359, 1056-1061, doi:10.1126/science.aan4829 (2018).
8 Wallrapp, A. et al. Erratum: The neuropeptide NMU amplifies ILC2-driven allergic lung inflammation. Nature 551, 658, doi:10.1038/nature24480 (2017).
9 Klose, C. S. N. et al. The neuropeptide neuromedin U stimulates innate lymphoid cells and type 2 inflammation. Nature 549, 282-286, doi:10.1038/nature23676 (2017).
10 Leung, D. Y. & Bieber, T. Atopic dermatitis. Lancet 361, 151-160, doi:10.1016/S0140-6736(03)12193-9 (2003).
11 McNeil, B. D. et al. Identification of a mast-cell-specific receptor crucial for pseudo-allergic drug reactions. Nature 519, 237-241, doi:10.1038/nature14022 (2014).
12 Usoskin, D. et al. Unbiased classification of sensory neuron types by large-scale single-cell RNA sequencing. Nature neuroscience 18, 145-153, doi:10.1038/nn.3881 (2015).
13 Marichal, T., Gaudenzio, N. et al. Guanine nucleotide exchange factor RABGEF1 regulates keratinocyte-intrinsic signaling to maintain skin homeostasis. J Clin Invest 126, 4497-4515, doi:10.1172/JCI86359 (2016).
14 Ando, T. et al. Mast cells are required for full expression of allergen/SEB-induced skin inflammation. J Invest Dermatol 133, 2695-2705, doi:10.1038/jid.2013.250 (2013).
15 Toyoda, M. et al. Nerve growth factor and substance P are useful plasma markers of disease activity in atopic dermatitis. Br J Dermatol 147, 71-79 (2002).
16 Salomon, J. & Baran, E. The role of selected neuropeptides in pathogenesis of atopic dermatitis. Journal of the European Academy of Dermatology and Venereology: JEADV 22, 223-228, doi:10.1111/j.1468-3083.2007.02399.x (2008).
17 Nawa, H., Kotani, H. & Nakanishi, S. Tissue-specific generation of two preprotachykinin mRNAs from one gene by alternative RNA splicing. Nature 312, 729-734 (1984).
18 Lattin, J. E. et al. Expression analysis of G Protein-Coupled Receptors in mouse macrophages. Immunome research 4, 5, doi:10.1186/1745-7580-4-5 (2008).
19 Sandor, K., Helyes, Z., Elekes, K. & Szolcsanyi, J. Involvement of capsaicin-sensitive afferents and the Transient Receptor Potential Vanilloid 1 Receptor in xylene-induced nocifensive behaviour and inflammation in the mouse. Neuroscience letters 451, 204-207, doi:10.1016/j.neulet.2009.01.016 (2009).
20 Gaudenzio, N. et al. Different activation signals induce distinct mast cell degranulation strategies. J Clin Invest 126, 3981-3998, doi:10.1172/JCI85538 (2016).
21 Subramanian, H. et al. PMX-53 as a dual CD88 antagonist and an agonist for Mas-related gene 2 (MrgX2) in human mast cells. Molecular pharmacology 79, 1005-1013, doi:10.1124/mol.111.071472 (2011).
22 Subramanian, H., Gupta, K., Guo, Q., Price, R. & Ali, H. Mas-related gene X2 (MrgX2) is a novel G protein-coupled receptor for the antimicrobial peptide LL-37 in human mast cells: resistance to receptor phosphorylation, desensitization, and internalization. J Biol Chem 286, 44739-44749, doi:10.1074/jbc.M111.277152 (2011).
23 Subramanian, H., Gupta, K. & Ali, H. Roles of Mas-related G protein-coupled receptor X2 on mast cell-mediated host defense, pseudoallergic drug reactions, and chronic inflammatory diseases. J Allergy Clin Immunol 138, 700-710, doi:10.1016/j.jaci.2016.04.051 (2016).
24 Fujisawa, D. et al. Expression of Mas-related gene X2 on mast cells is upregulated in the skin of patients with severe chronic urticaria. J Allergy Clin Immunol 134, 622-633 e629, doi:10.1016/j.jaci.2014.05.004 (2014).
25 Tatemoto, K. et al. Immunoglobulin E-independent activation of mast cell is mediated by Mrg receptors. Biochemical and biophysical research communications 349, 1322-1328, doi:10.1016/j.bbrc.2006.08.177 (2006).
26 Reber, L. L., Marichal, T. & Galli, S. J. New models for analyzing mast cell functions in vivo. Trends Immunol 33, 613-625 (2012).
27 Reber, L. L. et al. Imaging protective mast cells in living mice during severe contact hypersensitivity. JCI Insight 2, e92900, doi:10.1172/jci.insight.92900 (2017).
28 Kim, Y. S. et al. Central terminal sensitization of TRPV1 by descending serotonergic facilitation modulates chronic pain. Neuron 81, 873-887, doi:10.1016/j.neuron.2013.12.011 (2014).
29 Lansu, K. et al. In silico design of novel probes for the atypical opioid receptor MRGPRX2. Nat Chem Biol 13, 529-536, doi:10.1038/nchembio.2334 (2017).
30 Veiga-Fernandes, H. & Artis, D. Neuronal-immune system cross-talk in homeostasis. Science 359, 1465-1466, doi:10.1126/science.aap9598 (2018).

1. A method of treating a type 2 inflammation or a mast cell-dependent disease in a subject in need thereof, comprising
administering to the subject a therapeutically effective amount of an MRGPRX2 binding molecule.

2. The method according to claim 1, wherein the MRGPRX2 binding molecule is a MRGPRX2 inhibitor.

3. The method according to claim 2 wherein the type 2 inflammation encompasses type 2 inflammation of the skin, type 2 allergy and type 2 allergy of the skin.

4. The method according to claim 3 wherein the type 2 inflammation is a type 2 allergy of the skin.

5. The method according to claim claim 1, wherein the type 2 inflammation includes anaphylactic hypersensitivity, asthma, allergic rhinitis, atopic dermatitis, vernal conjunctivitis, eczema, IgE-mediated urticarial, food allergies, IgE-mediated anaphylaxis, esophagus eosinophilic and/or oesophagitis.

6. The method according to claim 4 wherein the type 2 allergy of the skin includes atopic dermatitis or eczema.

7. The method according to claim 6 wherein the type 2 allergy of the skin is an atopic dermatitis.

8. The method according to claim 1, wherein the MRGPRX2 binding molecule is mast-cell depleting antibody.

9. The method according to claim 1 or 7, wherein the mast-cell dependent disease is mastocytosis.

10. A method of treating a type 2 inflammation or mast cell-dependent disease in a subject in need thereof, comprising
administering to the subject a therapeutically effective amount of a therapeutic composition comprising a MRGPRX2 binding molecule.

11. (canceled)
